(19) United States
(12) Patent Application Publication
(10) Pub. No.: US 2011/0066232 A1
(43) Pub. Date: Mar. 17, 2011

(54) BIOPROSTHETIC VALVE HOLDER AND HANDLE WITH CUTTING MECHANISM AND METHOD OF USING SAME

(76) Inventors: Fernando A. Riveron, Wausau, WI (US); Donald D. Grover, Rothschild, WI (US); Fred P. Lane, Wausau, WI (US); Jan M. Pinkert Grover, Rothschild, WI (US)

(21) Appl. No.: 12/628,762

(22) Filed: Dec. 1, 2009

Related U.S. Application Data
(63) Continuation-in-part of application No. 12/422,797, filed on Apr. 13, 2009.
Application No. 12/422,797 is a continuation-in-part of application No. 12/272,514, filed on Nov. 17, 2008.
(60) Provisional application No. 61/119,130, filed on Dec. 2, 2008.
(60) Provisional application No. 61/044,483, filed on Apr. 12, 2008.
(60) Provisional application No. 60/988,296, filed on Nov. 15, 2007.

(51) Int. Cl. A61F 2/24; A61B 17/10

(52) U.S. Cl. 623/2.11; 606/138

(57) ABSTRACT

A surgical apparatus for use with a prosthetic holder to which a prosthesis is secured by sutures. The apparatus includes an elongated handle having a grip end and a coupler end and defining a first aperture extending along a length from the grip end to the coupler end. The apparatus can also include a first shaft at least partially disposed within the first aperture with a first actuator at the grip end and a holder coupling mechanism at the coupler end. The apparatus can have one of a second shaft and a sheath disposed along the handle with a second actuator at the grip end and a cutting element at the coupler end. A force applied to the first actuator enables one of engagement and disengagement with the prosthesis holder, and a force applied to the second actuator enables deployment of a cutting element to cut the sutures securing the prosthesis to the prosthetic holder.

### RELATED APPLICATIONS

This application claims priority to U.S. Provisional Patent Application No. 61/119,130, filed on Dec. 2, 2008, U.S. patent application Ser. No. 12/422,797, filed on Apr. 13, 2009, which claims priority to U.S. Provisional Patent Application No. 61/044,483, filed on Apr. 12, 2008, and U.S. patent application Ser. No. 12/272,514, filed on Nov. 17, 2008, which claims priority to U.S. Provisional Patent Application No. 60/988,296, filed on Nov. 15, 2007, the entire contents of all these earlier patent applications being hereby incorporated by reference.

### BACKGROUND

The present invention relates to implements for facilitating minimally invasive surgical procedures, and more particularly for cardiac valve replacement surgery.

### SUMMARY

In some embodiments, the present invention provides a surgical apparatus for use with a prosthetic holder to which a prosthesis is secured by sutures. The apparatus can include an elongated handle having a grip end and a coupler end and defining a first aperture extending along a length from the grip end to the coupler end. The apparatus can also include a first shaft at least partially disposed within the first aperture with a first actuator at the grip end and a holder coupling mechanism at the coupler end. The apparatus can have one of a second shaft and a sheath disposed along the handle with a second actuator at the grip end and a cutting element at the coupler end. A force applied to the first actuator enables one of engagement and disengagement with the prosthesis holder, and a force applied to the second actuator enables deployment of a cutting element to cut the sutures securing the prosthesis to the prosthetic holder.

Another embodiment of the invention provides a surgical apparatus for use with a prosthesis including a prosthetic holder having a handle coupling element, the prosthetic holder coupled to a prosthesis by sutures. The apparatus can also include an elongated handle having a grip end and a coupler end, the handle defining a first aperture extending along a length from the grip end to the coupler end, a first shaft at least partially disposed within the first aperture and having a first actuator at the grip end and a holder coupling mechanism at the coupler end, and one of a second shaft and a sheath disposed along the handle and having a second actuator at the grip end and a cutting element at the coupler end. A force applied to the first actuator can enable one of engagement and disengagement of the holder coupling mechanism with the handle coupling element of the prosthesis holder, and a force applied to the second actuator can enable deployment of a cutting element to cut the sutures securing the prosthesis to the prosthetic holder when the prosthesis holder is engaged.

Still another embodiment of the present invention provides a method of performing a surgical procedure in which is used a prosthetic holder and an elongated handle having a holder coupling mechanism and a cutting mechanism at a coupler end and a holder coupling actuator and a cutting actuator at a grip end. The method can include tying a prosthesis to the prosthetic holder with sutures, coupling the prosthetic holder to a handle by actuating the holder coupling mechanism, positioning the prosthesis with the grip end of the handle, and decoupling the prosthetic holder from the handle by actuating the holder coupling mechanism. The method can also include suturing the prosthesis to the surrounding tissue, recoupling the prosthetic holder from the handle by actuating the holder coupling mechanism, and cutting the sutures by actuating the cutting mechanism.

Other aspects of the present invention will become apparent by consideration of the detailed description and accompanying drawings.

### BRIEF DESCRIPTION OF THE DRAWINGS

FIG. 1A is a side view of a latch type surgical apparatus according to an embodiment of the present invention, shown with the cutter retracted;

FIG. 1B is a cross-sectional side view of the latch type surgical apparatus illustrated in FIG. 1A, shown with the cutter retracted;

FIG. 2A is a side view of the latch type surgical apparatus of FIGS. 1A and 1B, shown with the cutter extended;

FIG. 2B is a cross-sectional side view of the latch type surgical apparatus illustrated in FIGS. 1A-2A, shown with the cutter extended;

FIG. 3 is a perspective view of a latch type surgical apparatus according to an embodiment of the present invention, shown disconnected from a socket type prosthetic holder with a valve prosthesis attached;

FIG. 4 is a side view of a portion of a latch type surgical apparatus according to an embodiment of the present invention, shown disconnected from a socket type prosthetic holder;

FIG. 5A is a cross-sectional view of a portion of a latch type surgical apparatus according to an embodiment of the present invention, shown connected to a socket type prosthetic holder and with the cutter retracted;

FIG. 5B is a cross-sectional view of the portion of the latch type surgical apparatus illustrated in FIG. 5A, shown connected to a socket type prosthetic holder and with the cutter extended;

FIG. 6 is a perspective view of a socket type prosthetic holder according to an embodiment of the present invention, shown in an open position with a valve prosthesis;

FIGS. 7A-7D are respectively, bottom, side, top, and detailed views of a socket type prosthetic holder according to an embodiment of the present invention, shown in an open position;

FIG. 8 is a perspective view of a socket type prosthetic holder according to an embodiment of the present invention, shown in a closed position;

FIGS. 8A-8D are respectively, bottom, side, top, and detailed views of the socket type prosthetic holder illustrated in FIG. 8, shown in a closed position;

FIG. 9A is a side view of a socket type surgical apparatus according to an embodiment of the present invention, shown disconnected from a knob type prosthetic holder;

FIG. 9B is cross-sectional side view of the surgical apparatus shown in FIG. 9A;

FIGS. 9C and 9D are respectively, a perspective view and a side view of a portion of a socket type surgical apparatus according to an embodiment of the present invention, shown disconnected from a knob type prosthetic holder;

FIG. 10 is a perspective view of a knob type prosthetic holder according to an embodiment of the present invention, shown in an open position and with a valve prosthesis;

FIGS. 11A-11D are respectively, bottom perspective, bottom, side, and top views of the knob type prosthetic holder illustrated in FIG. 10, shown in an open position;

FIGS. 12A-12E are respectively, bottom perspective, bottom, side, top, and detail views of a knob type prosthetic holder according to an embodiment of the present invention, shown in a closed position;

FIG. 13 is a side view of a claw type surgical apparatus according to an embodiment of the present invention, shown disconnected from a knob type prosthetic holder;

FIG. 13A is a cross-sectional view of the surgical apparatus shown in FIG. 13, taken at 13-13 of FIG. 13;

FIG. 14 is a top view of the claw type surgical apparatus shown in FIGS. 13 and 13A, shown disconnected from a knob type prosthetic holder;

FIG. 14A is a cross-sectional view of the surgical apparatus shown in FIGS. 13-14, taken at 14-14 of FIG. 14;

FIG. 15 is an exploded perspective view of the claw type surgical apparatus shown in FIGS. 13-14A, shown with a knob type prosthetic holder;

FIGS. 16A-16E are respectively, side, proximal end, distal end, cross-sectional, and perspective views of an outer blade guard of a claw type surgical apparatus according to an embodiment of the present invention;

FIGS. 17A-17E are respectively, side, proximal end, distal end, cross-sectional, and perspective views of a blade mount of a claw type surgical apparatus according to an embodiment of the present invention;

FIGS. 18A-18E are respectively, side, proximal end, distal end, cross-sectional, and perspective views of a blade of a claw type surgical apparatus according to an embodiment of the present invention;

FIGS. 19A-19E are respectively, side, proximal end, distal end, cross-sectional, and perspective views of a claw sleeve of a claw type surgical apparatus according to an embodiment of the present invention;

FIGS. 20A-20D are respectively, side, proximal end, distal end, and perspective views of a grasping claw of a claw type surgical apparatus according to an embodiment of the present invention;

FIGS. 21A-21D are respectively, side, proximal end, cross-sectional, and perspective views of a prosthetic holder according to an embodiment of the present invention and usable with the claw type surgical apparatus of FIGS. 13-20;

FIGS. 22A and 22B are respectively, side and cross-sectional views of a coupler end of a claw type surgical apparatus according to an embodiment of the present invention, shown disconnected from a knob type prosthetic holder;

FIGS. 23A and 23B are respectively, side and cross-sectional views of a coupler end of a claw type surgical apparatus according to an embodiment of the present invention, shown with the claw extended to connect with a knob type prosthetic holder embodiment;

FIGS. 24A and 24B are respectively, side and cross-sectional views of a coupler end of a claw type surgical apparatus according to an embodiment of the present invention, shown connected to a knob type prosthetic holder;

FIGS. 25-28 illustrate various valve prosthetics secured to various prosthetic holder embodiments compatible with types of surgical apparatuses disclosed herein.

### DETAILED DESCRIPTION

Before any embodiments of the present invention are explained in detail, it is to be understood that the present invention is not limited in its application to the details of construction and the arrangement of components set forth in the following description or illustrated in the accompanying drawings. The present invention is capable of other embodiments and of being practiced or of being carried out in various ways.

The human heart has both bicuspid and tricuspid valves—valves with two and three leaflets, respectively—which regulate blood flow through the heart. If either type of heart valve is not functioning properly, it may necessitate replacement with a bioprosthetic valve (e.g., a valve from a human donor cadaver, a porcine valve, or a biomechanical valve element). Alternatively, a ring or band can be implanted to repair a poorly functioning valve. In order to provide superior outcomes for surgical valve replacement or repair (improve heart function, decrease recovery time, etc.), it is important to reduce the invasiveness of the procedure. A surgical apparatus for positioning the replacement valve or repair ring or band remotely through a small incision can reduce trauma to the heart and connecting vessels. While the majority of the description of the present invention that follows refers by example to a replacement valve, the disclosed invention is capable of being used in surgical procedures involving various types of replacement valves and valve repair elements including rings and bands.

A valve prosthesis 410 (see, for example, FIG. 6) includes a generally cylindrical base 412 from which leaflets of the valve extend radially inward. An attachment ring 418 is connected around the outer circumference of the base 412 to attach and seal the valve prosthesis 410 to an aortic wall, usually by suturing. Commissure support struts 414 extend axially from the base 412 to maintain the positioning and seal of the prosthesis 410 with respect to surrounding heart tissue, and are elastically deflectable with respect to the base 412. The elasticity allows the support struts 414 to be deflected radially inward while the prosthesis 410 is being implanted to provide the surgeon with better access to the attachment ring 418 and surrounding heart tissue for suturing.

In some embodiments, a surgical apparatus for valve prostheses can consist of a prosthetic holder 430 connected to a longitudinally extending handle 500. The handle 500 can have a contoured grip 506 at one end (grip end) and a holder coupling mechanism at the opposing end (coupler end). Various embodiments of the handle 500 presented in the figures are discussed in further detail below.

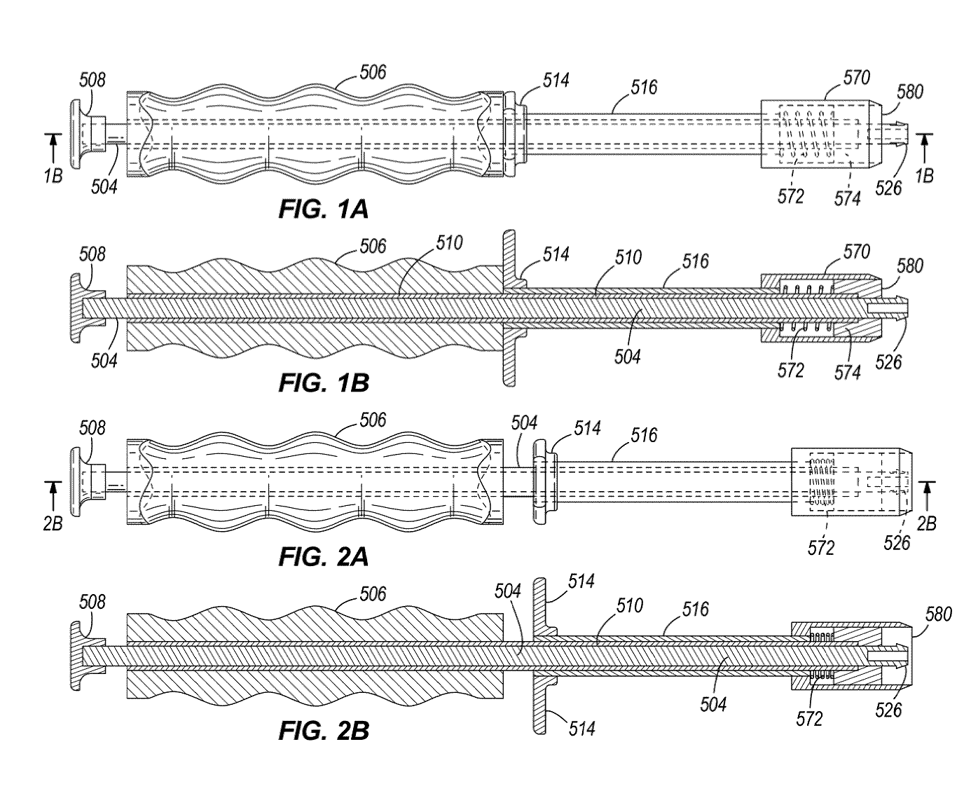

As shown in FIGS. 1A-2B, a shaft 504 can extend through a sleeve 510 of the handle 500. The end of the holder shaft 504 adjacent the grip end of the handle 500 can include an actuator button 508. Alternatively, an actuator can be coupled to the shaft 504 and extend through an aperture at any point along or adjacent the grip 506 of the handle 500. The opposing end of the shaft 504 can include a holder coupling mechanism, which in the embodiment of FIGS. 1A-5B, comprises a finger 524 with a latch element 526. The latch element 526, which can be spring-activated, is received in mating engagement with a handle coupling mechanism of the prosthetic holder 430. A relief 522 can be positioned along the finger 524 to allow some flexibility of the holder coupling mechanism.

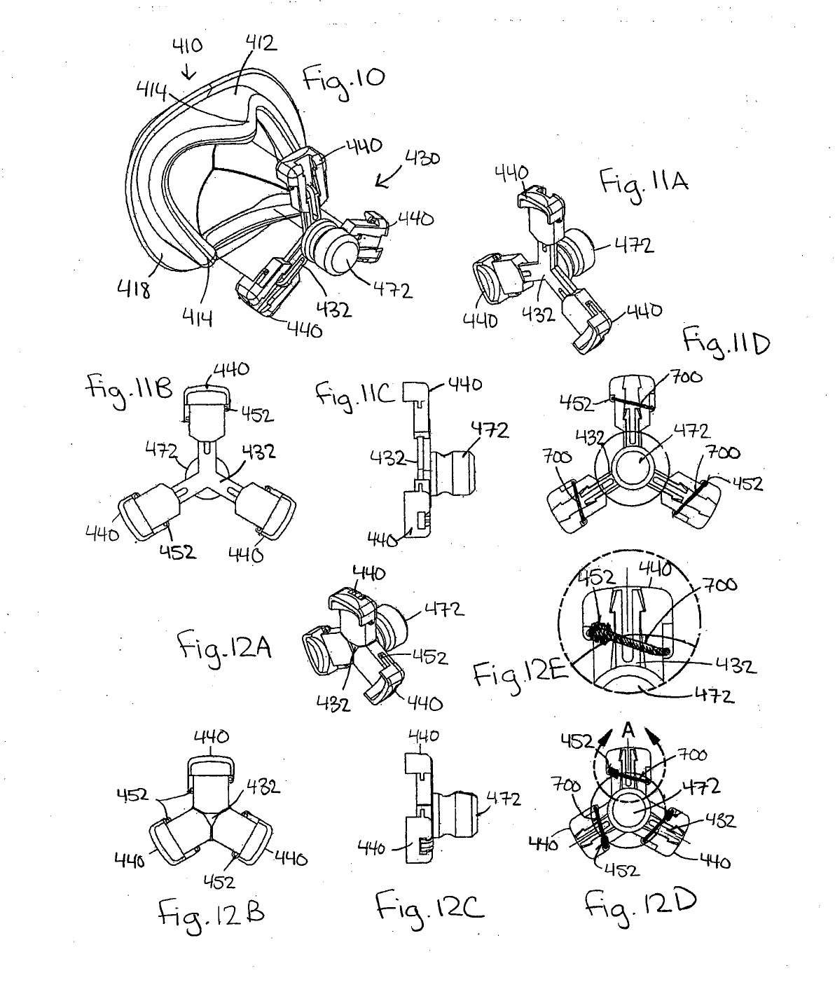

The latch element 526 can be activated by the actuator button 508 to engage or disengage the handle coupling mechanism of the holder 430. In some embodiments, the holder coupling mechanism can be actuated to provide movement of the prosthetic holder 430 with respect to the handle 500 into configurations shown in FIGS. 7-8 and 11-12. In some embodiments, two actuators associated with the holder coupling mechanism can be provided—one for each function. Alternatively, a single actuator (as shown in the illustrated embodiments) can be actuated in various ways to provide various functions. For example, a push or pull of the actuator button 508 can enable engagement or disengagement of the handle 500 with the holder 430, while a twist or rotation of the actuator button 508 can provide relative movement of the holder 430 with respect to the handle 500, or vise versa.

The handle 500 can also include a cutter mechanism 516, positioned circumferentially around the sleeve 510 over the shaft 504 at the coupler end of the handle 500. The cutter mechanism 516 can include an actuator 514 positioned adjacent the grip 506, and a cutter 570 with a blade edge 580 at the opposing end. The cutter 570 can be biased to a retracted position (FIGS. 1A and 1B) by a retraction spring 572. The actuator 514 enables the cutter mechanism 516 to be extended axially (FIGS. 2A and 2B) against the spring bias in order to provide the cutting function, which will be discussed in further detail below. A retraction guide 574 positioned at the coupler end of the sleeve 510 can provide guidance for the cutter 570 and a stop for the spring 572.

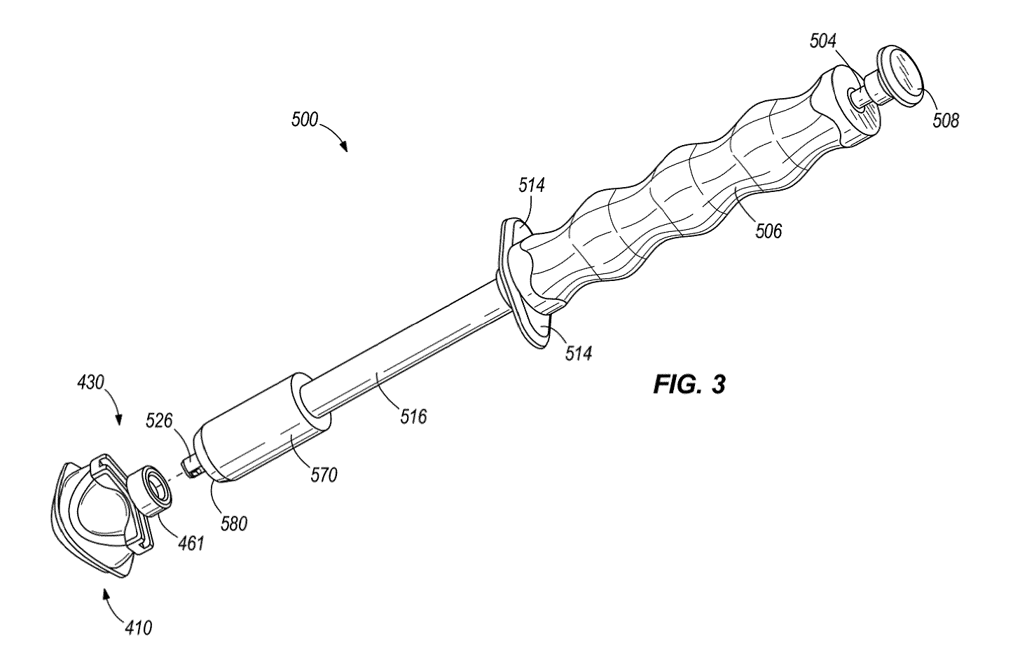

As shown in FIGS. 3-6, a prosthetic holder 430 is designed to provide an interface between the valve prosthesis 410 and the handle 500. Consequently, the holder 430 includes a handle coupling mechanism which enables selective connection to the holder coupling mechanism of the handle 500. In the embodiment of FIGS. 3-8D, the handle coupling mechanism of the prosthetic holder 430 includes a socket 461 contoured to receive the latch element 526 of the handle 500. The socket 461 is positioned centrally with respect to a branched retraction guide 432. Each of the branches extends to a reciprocating face 442 and terminates at a retaining face 440.

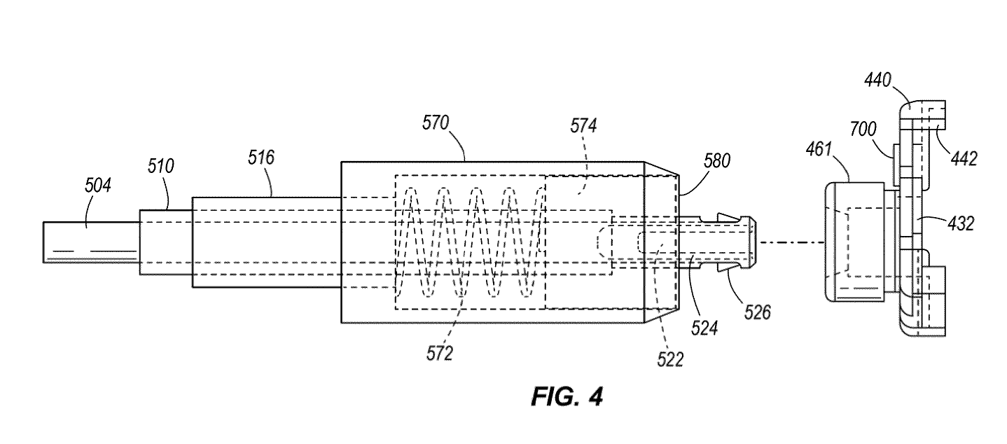

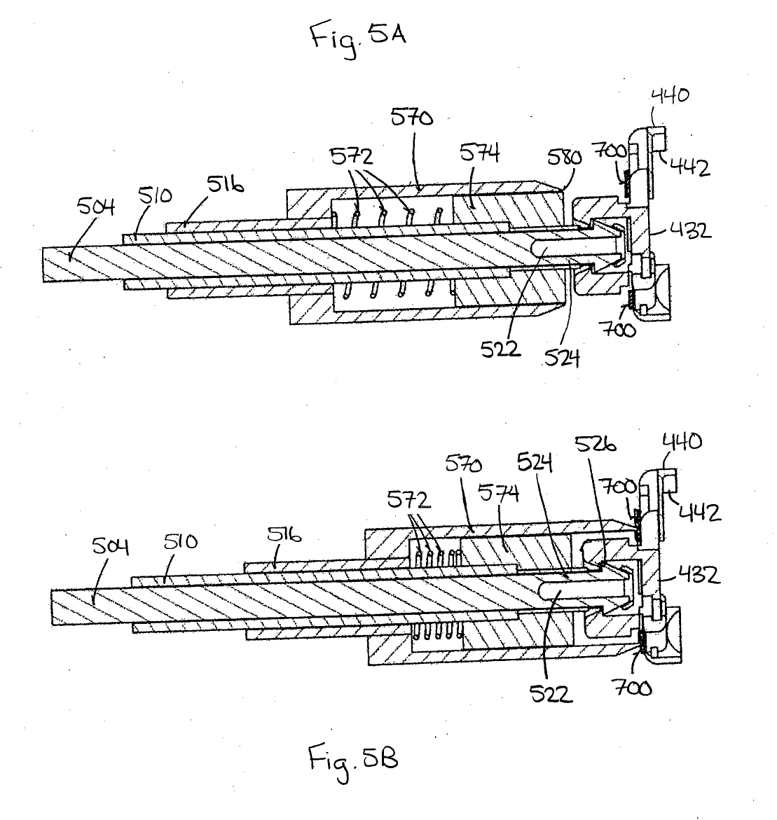

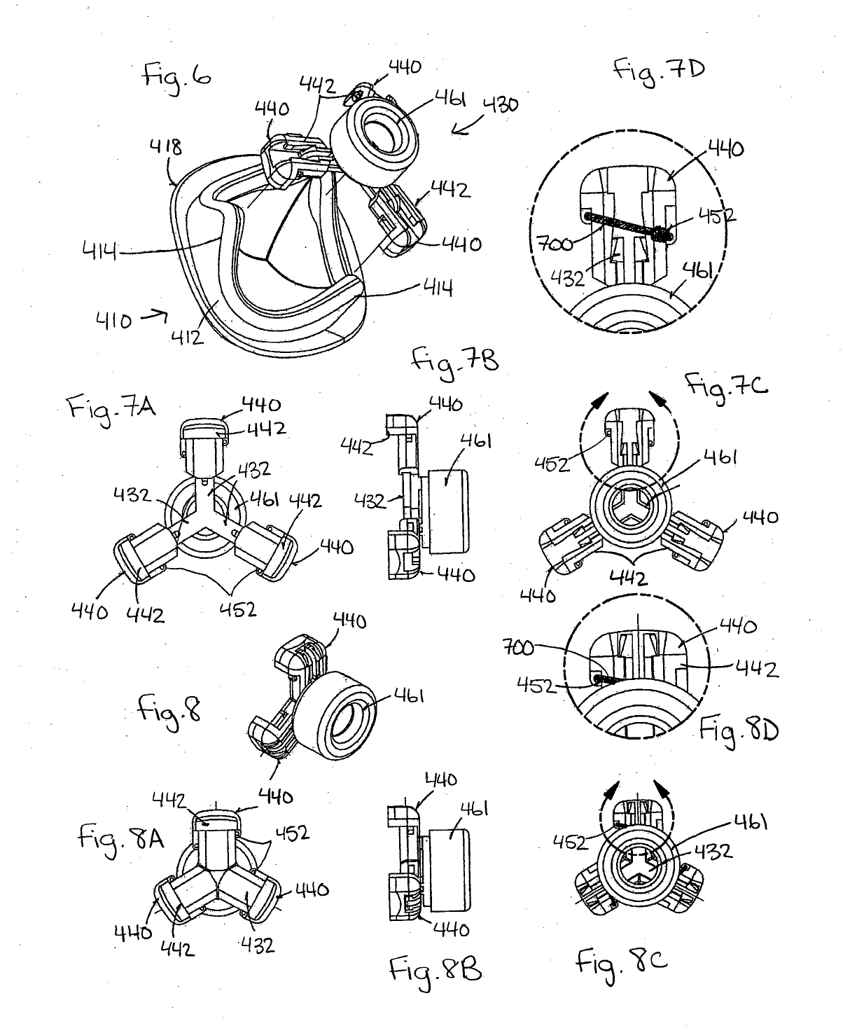

A support strut 414 of the valve prosthesis can be secured between the reciprocating and retaining faces 442,440 of each branch and held in place by sutures 700 as shown in FIGS. 4-5B, 7D, and 8D. A suture eyelet 452 can be provided on each side of the reciprocating and/or retaining face 442, 440 of each branch for the suture 700 to be tied through to anchor the support strut 414. The reciprocating and retaining faces 442, 440 and secured support strut 414 can be retracted radially along the retraction guide 432 from an open position (FIGS. 7-7D) to a closed position (FIGS. 8-8D). This retraction can be performed by direct manual manipulation of the holder 430 or indirectly by operation of an actuator on the handle 500, as previously described. The ability to retract the support struts 414 of the valve prosthesis 410 provides a surgeon better access to the attachment ring 418 for suturing the prosthesis 410 to surrounding tissue.

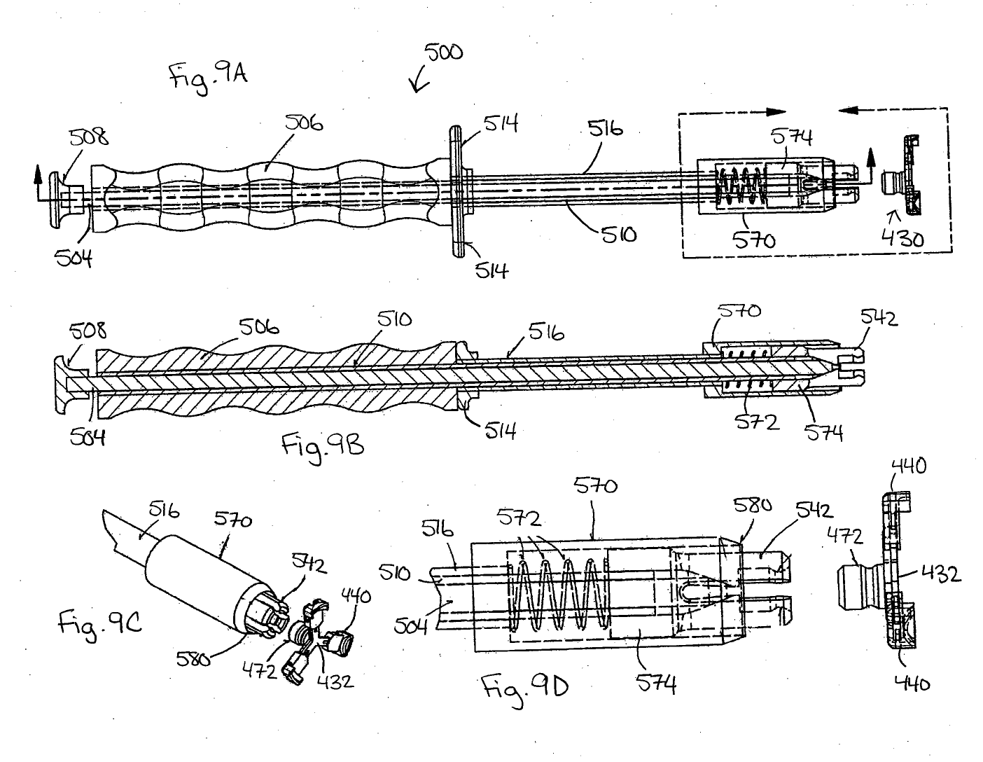

FIGS. 9A-9D illustrate a prosthetic valve holder handle 500 with an alternative type of holder coupler mechanism. Specifically, the latch element 526 of the previous handle embodiment can be replaced with a socket 542. FIGS. 10-12E illustrate an embodiment of the prosthetic holder 430 adapted to selectively connect to the socket type handle embodiment. In this alternative embodiment, the socket 461 of the previous holder embodiment can be replaced with a knob 472. As shown in FIGS. 10-12E, the prosthetic holder 430 can alternatively have only one of a reciprocating face 442 and a retaining face 440 for each branch, as the suture eyelets 452 provide sufficient structure to anchor the support strut 414 to the holder 430 with a suture 700.

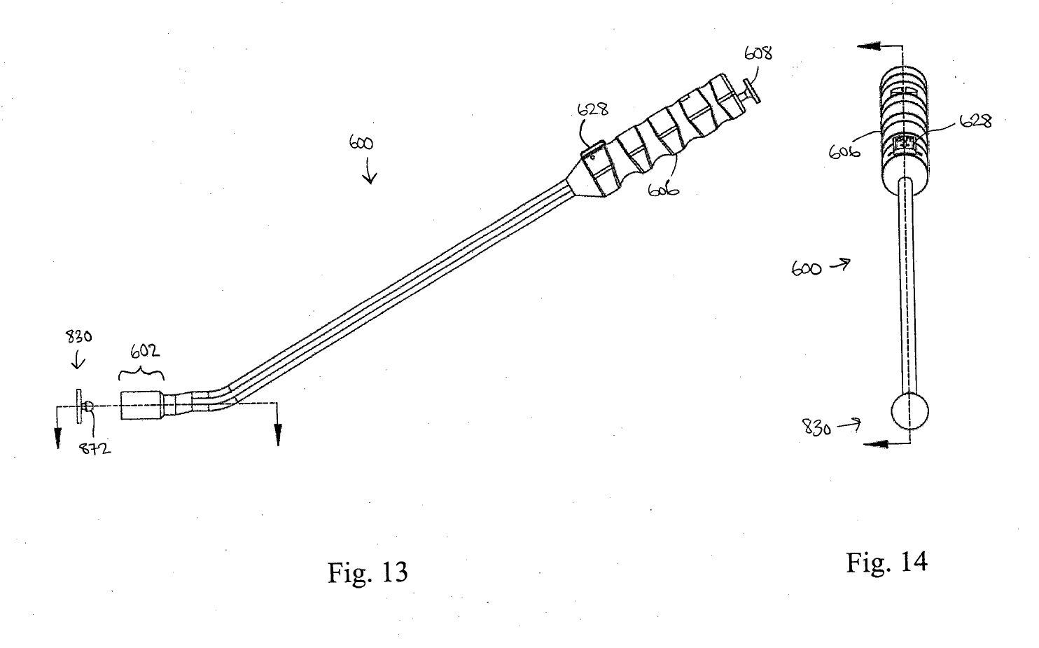

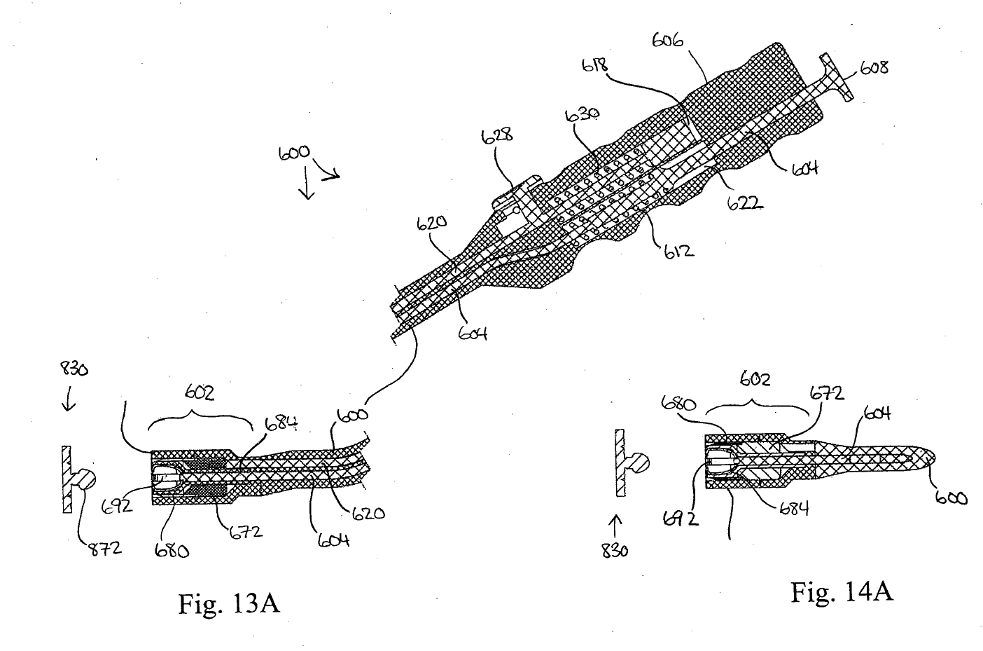

In another alternative embodiment of the present invention illustrated in FIGS. 13-15, an elongated handle 600 can have a contoured grip 606 at one end (grip end) and a holder engaging assembly 602 at an opposing end (coupler end). For discussion purposes, elements of the surgical apparatus of the embodiment illustrated in FIGS. 13-24B will be described with “proximal” referring to the location, portion, or perspective closest to the grip end of the handle and “distal” referring to the location, portion, or perspective farthest from the grip end.

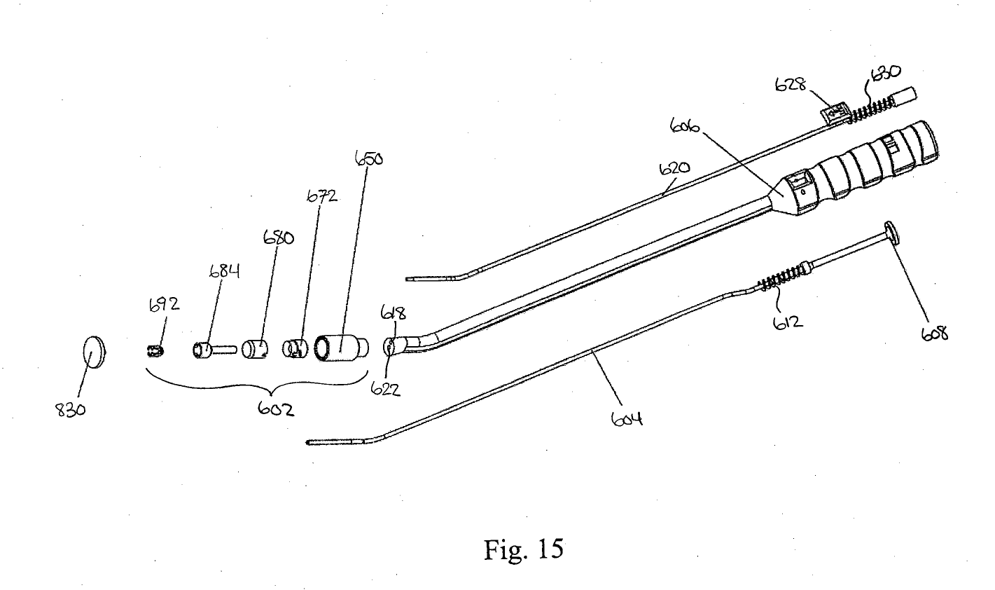

As shown in FIGS. 13 and 15, some embodiments of a handle 600 can be provided with a bend between the grip and coupler ends, which can make the surgical apparatus more ergonomically functional. A first aperture 622 can be defined longitudinally through the handle 600 from the grip end to the coupler end, and a first shaft 604 can be positioned within the first aperture 622. In some embodiments, the first shaft 604 can be formed of flexible plastic or steel braided cable. A proximal end of the first shaft 604 extending from the grip end can terminate in or be coupled to a first actuator 608. A distal end of the first shaft 604 can terminate in or be coupled to a holder coupling mechanism 692. In the illustrated embodiment of FIGS. 13-24B, the coupling mechanism 692 is a grasping claw.

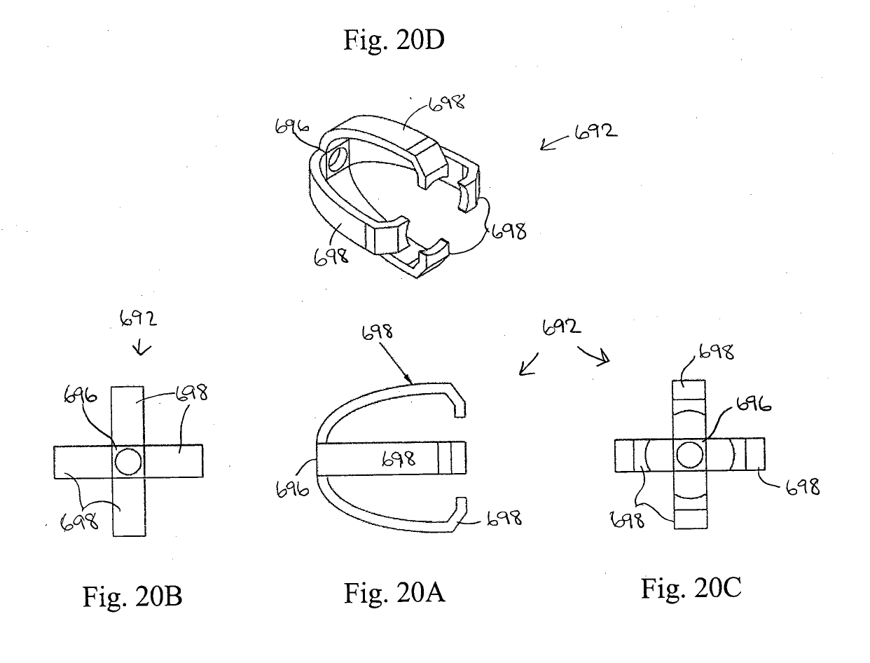

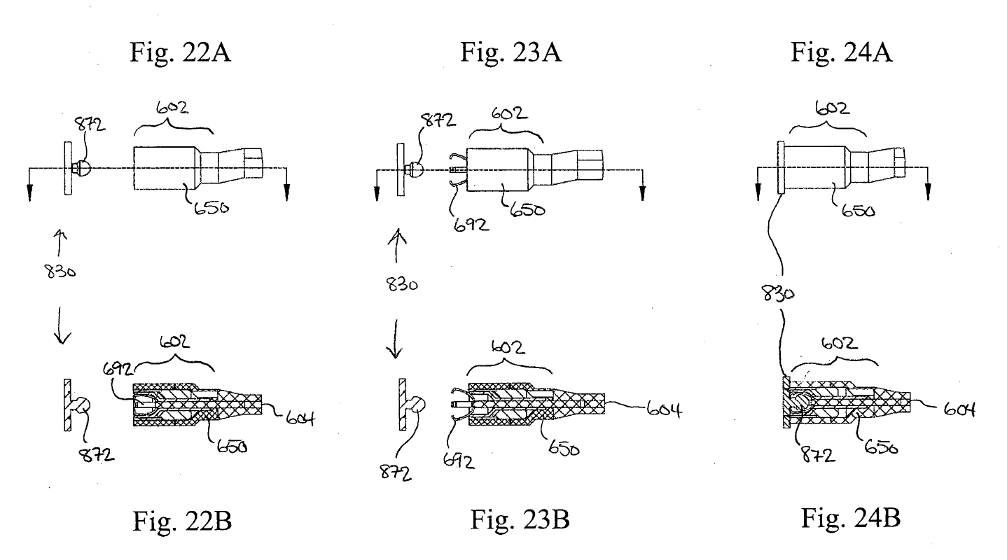

Shown in more detail in FIGS. 20A-20D, the illustrated grasping claw 692 includes multiple resiliently flexible arms 698 connected at a joint 696. In some embodiments, the grasping claw 692 can be formed of spring steel or spring polymer. As illustrated in FIGS. 13A and 14A, the distal end of the first shaft 604 can be connected to the grasping claw 692 at or near the joint 696. Each arm 698 can include at least one retaining finger 694 at its distal end, which can extend at an angle radially inward from the arm 698. As illustrated in FIGS. 22B and 24B, the arms 698 of the grasping claw 692 can be flexed radially inward, reducing the diameter of the claw 692, in order for it to fit within the holder engaging assembly 602. When the grasping claw 692 is not retained by the holder engaging assembly 602, as shown in FIG. 23B, the arms 698 return to an unflexed state, thereby increasing the diameter of the claw 692. In the unflexed state, the claw 692 can receive a knob 872 of a prosthetic holder 830. FIGS. 22A-24B illustrate the flexed and unflexed states of the claw 692, the control of which is explained below.

The first aperture 622 can include a wider diameter portion to accommodate a first biasing element 612, which can abut a flange on the first shaft 604 to bias the shaft in a proximal direction. Consequently, the grasping claw 692 can be maintained within the holder engaging assembly 602 in a closed or retracted state without applying any external force. In order to move the grasping claw 692 out of the holder engagement assembly 602 and return the arms 698 to the unflexed state, a force can be exerted on the first actuator 608 to overcome the bias and move the first shaft 604 distally. A locking mechanism can be associated with the first shaft 604 to prevent unintentional movement of the grasping claw 692.

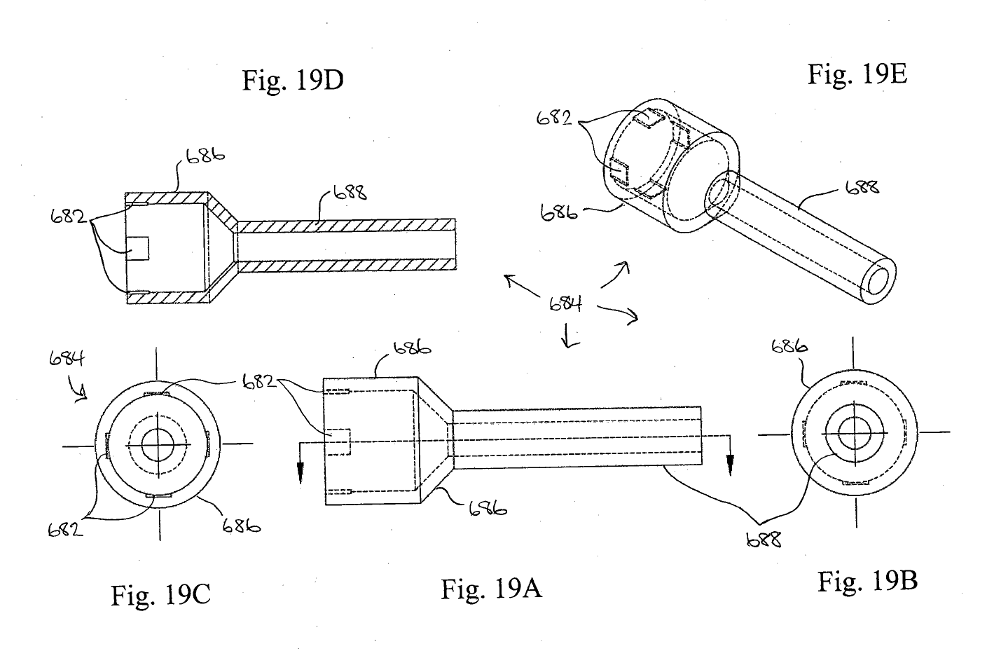

The grasping claw 692 and the connecting portion of the first shaft 604 are surrounded within the holder engagement assembly 602 by a claw sleeve 684. As illustrated in FIGS. 19A-19E, the claw sleeve 684 can include a sheath 688 through which the first shaft 604 extends, and a receptacle 686 sized to receive the grasping claw 692. Recesses 682 can be defined in the inner surface of the receptacle 686 at the distal end of the claw sleeve 684. Each recess 682 can be positioned to receive an arm 698 of the grasping claw 692 and guide the opening and closing motion of the claw as it is expelled from or drawn into the holder engagement assembly 602.

A second aperture 618 can be defined longitudinally through the handle 600 from the grip end to the coupler end, and a second shaft 620 can be positioned within the second aperture 618. In some embodiments, the second shaft 620 can be formed of flexible plastic or steel braided cable. A proximal end of the second shaft 620 extending from the grip end can include or be coupled to a second actuator 628. A distal end of the second shaft 620 can terminate in or be coupled to a blade mount 672. The blade mount 672 couples a blade element 680 to the second shaft 620.

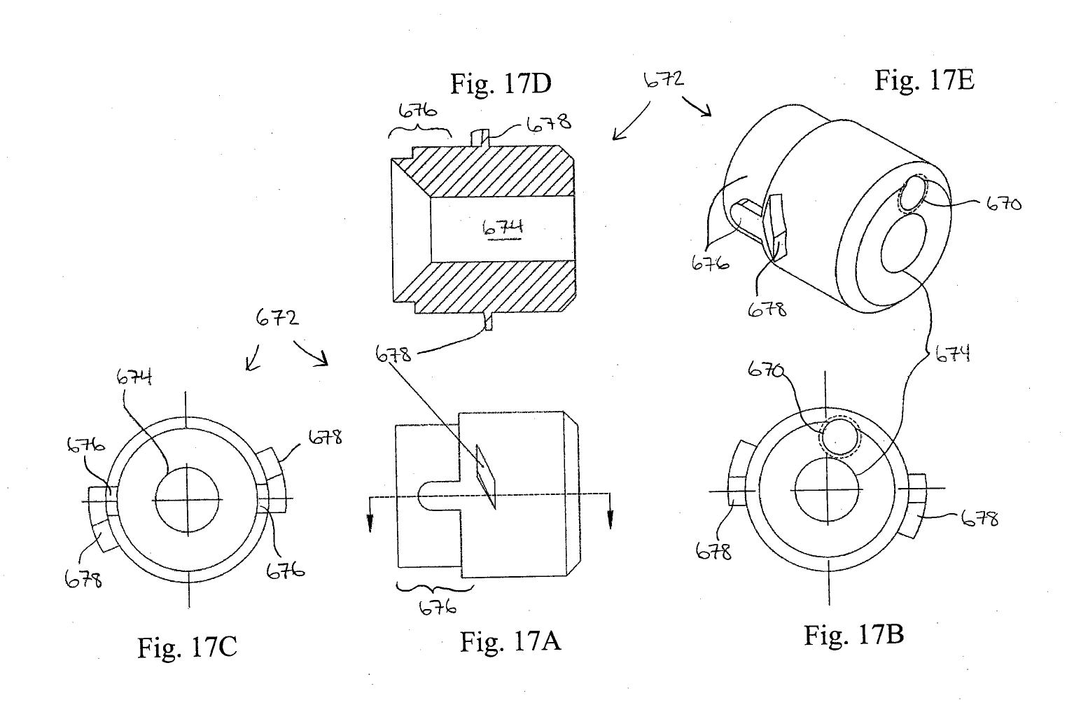

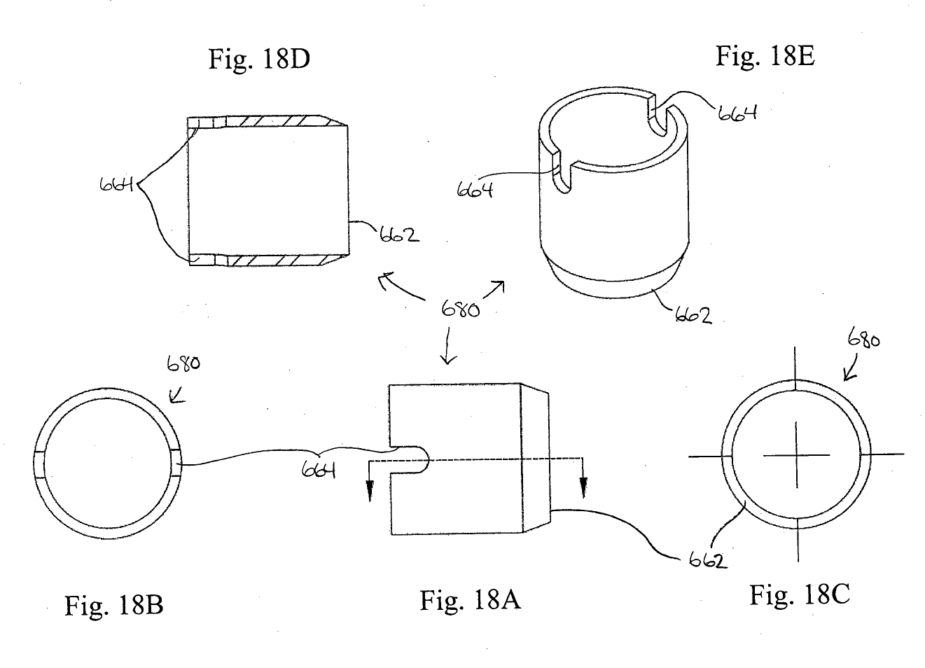

As shown in more detail in FIGS. 17A-17E, the blade mount 672 can be coupled to the second shaft 620 at a connector 670. The connector 670 can be offset from the central axis of the blade mount 672 in order to accommodate an aperture 674, which accommodates the claw sleeve 684. The blade mount 672 can include a blade coupler 676, which can provide support for and prevent rotation of the blade element 680 with respect to the blade mount 672. The blade mount 672 can also include posts 678, the purpose of which will be discussed below. The blade element 680, shown in detail in FIGS. 18A-18E, can include a mount coupler 664, to secure the blade element 680 in place on the blade mount 672 and prevent relative movement between the elements. The blade element 680 further includes a blade edge 662 capable of cutting sutures 700.

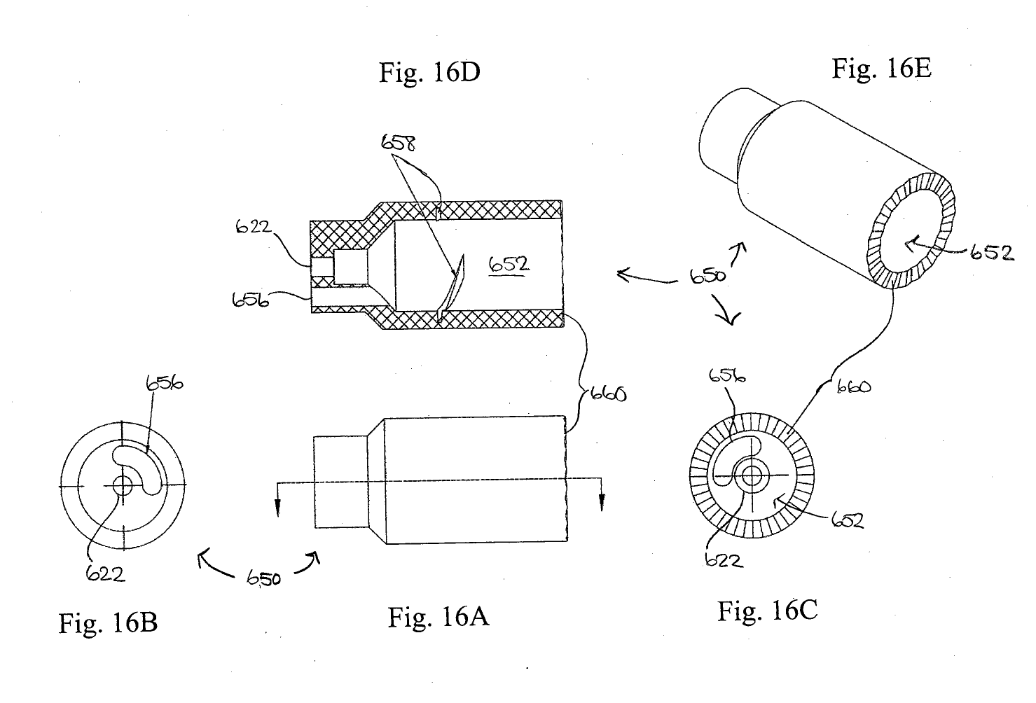

A blade guard 650, illustrated in detail in FIGS. 16A-16E, can be provided at the distal end of the handle 500, to protect tissue in the surgical field from the blade element 680. The blade guard 650 can include an aperture 652 extending from its distal end, which accommodates the blade mount 672 and blade element 680. The first aperture 622 of the handle 600 can extend from the proximal end of the blade guard 650 and connect to aperture 652. An arcuate aperture 656 can also extend from the proximal end of the blade guard 650 and connect to aperture 652. The arcuate aperture 656 can be radially aligned with the second aperture 618 of the handle 600 and the connector 670 of the blade mount 672. The inner surface of aperture 652 can include a track 658, which can be positioned to receive the posts 678 of the blade mount 672. As shown in FIG. 16D the track 658 can provide a helical path for the posts 678. The distal end of the blade guard 650 can include teeth 660 which can engage teeth 832 of a prosthetic holder 830 to prevent rotation of the holder 830 with respect to the handle 600.

The second aperture 618 of the handle 600 can include a wider diameter portion to accommodate a second biasing element 630, which can abut a flange on the second shaft 620 to bias the second shaft 620 in a proximal direction. Consequently, the blade element 680 can be maintained within the holder engaging assembly 602 in a retracted state without applying any external force. In order to move the blade element 680 out of the holder engagement assembly 602 to engage and cut a suture 700, a force can be exerted on the second actuator 628 to overcome the bias and move the second shaft 620 distally. A locking mechanism can be associated with the second shaft 620 to prevent unintentional movement of the blade element 680.

When the second shaft 620 is moved distally, the posts 678 of the blade mount 672 follow the helical path of the post tracks 658, thereby imparting a rotational motion to the blade mount 678 and blade element 680 as they move distally. In this way, the blade element can be deployed from the holder engaging assembly to cut sutures 700 which secure a valve prosthesis to a prosthetic holder 830. The arcuate aperture 656 of the blade guard 650 enables the second shaft 620 to follow the rotational motion of the blade mount 672 to which it is connected. Alternatively, one or more posts or similar protrusions can be provided within the blade guard 650 to be received in one or more tracks defined by the blade mount 672. In a further alternative embodiment, the one or more posts and tracks can be provided or defined by the outer surface of the claw sleeve 684 and an inner surface of the blade mount 672.

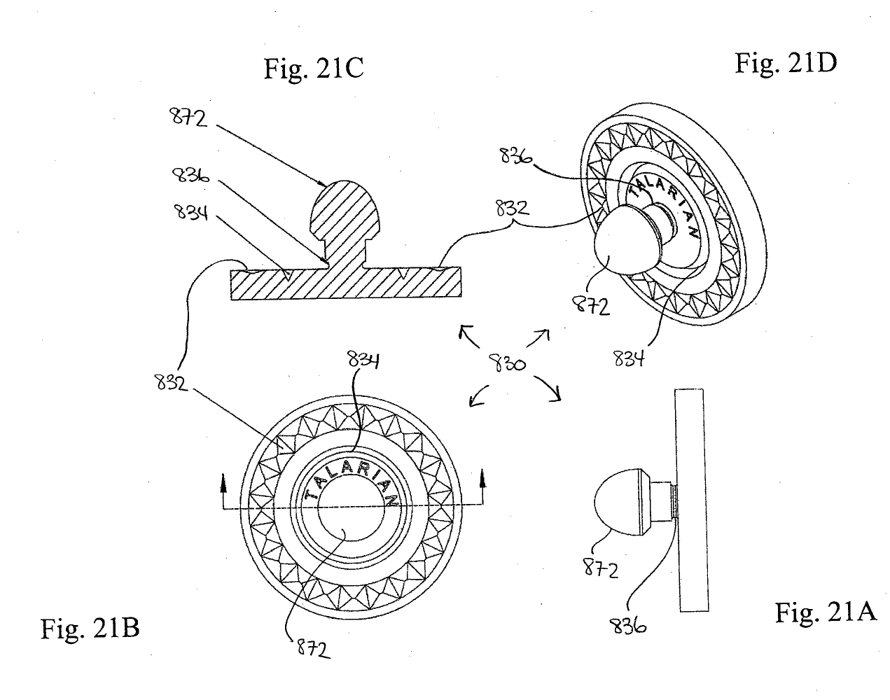

One embodiment of a prosthetic holder 830 usable with the grasping type handle embodiment 600 is shown in detail in FIGS. 21A-21D. The holder 830 can include a knob 872 designed to be selectively engaged by the grasping claw 692 and providing an anchor around which sutures 700 can be tied to secure a prosthesis 810 to the holder 830. A suture channel 836 can be provided at the base of the knob 872 to retain sutures 700 which secure prosthesis 810 to the holder 830. As previously mentioned, the holder 830 can also include teeth 832 arranged to engage teeth 660 of the blade guard 550 and prevent rotational motion of the holder 830 with respect to the handle 600. A cutting channel 834 can be positioned radially between the knob 872 and the teeth 832. The cutting channel 834 can receive the blade edge 662 when the blade element 830 is deployed to cut the sutures 700 that extend across it.

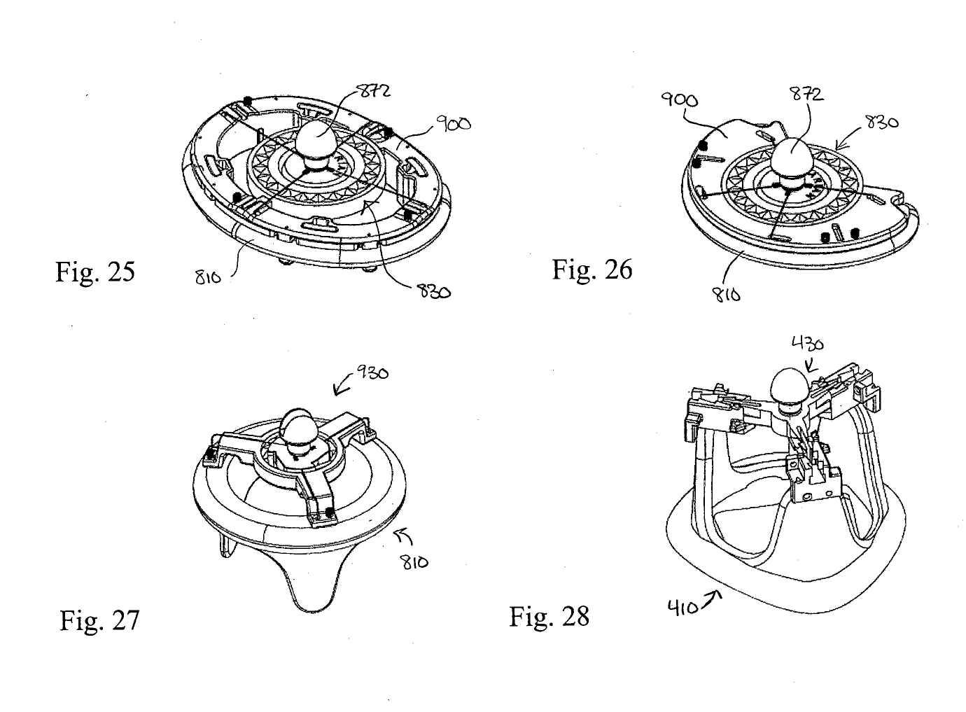

FIGS. 25 and 26 illustrate two sample integrations of the prosthetic holder 830 usable with the grasping type handle embodiment 600. Two types of adapters 900 can enable the holder 830 to be coupled to ring and band prostheses 810. FIGS. 27 and 28 show two alternative prosthetic holders 930, 430 for use with different types of prostheses, which are also usable with the grasping type handle embodiment 600.

One embodiment of a surgical procedure utilizing the prosthesis holder and handle with cutting mechanism can be performed as follows. A valve prosthesis 810 can secured to a prosthetic holder 830 by sutures 700 tied from the base of the prosthesis to the suture channel 836 around the knob 872. A force can be applied to the first actuator 608 deploying the grasping claw 692 from the coupler end of the handle 600. The positioning of the first actuator 608 on the end of the grip 606 can allow a surgeon or surgical assistant to apply this force with a single finger and without necessitating adjustment of the handle 600 within his or her hand. The grasping claw 692 can positioned around the knob 872, and the force removed from the first actuator 608 causing the grasping claw 692 to engage the knob 872 of the holder 830 as it retracts into the coupling end of the handle 600. Depending on the type of prosthesis and holder used, a cinching mechanism of the holder 430 can be used to retract support struts 414 and expose the attachment ring 418 (see FIGS. 7-9 and 10-12).

The valve prosthesis 810, holder 830, and holder engaging assembly 602 can be positioned through an incision by the surgeon or assistant using the handle 600. When the valve is in the desired position, a force can again applied to the first actuator 608 to deploy the grasping claw 692 and release the holder 830 and prosthesis 810. The handle 600 can then be removed from the surgical site so that the surgeon can suture the valve into place in the heart. After the valve prosthesis 810 is secured, the handle 600 can again be inserted through the incision and the grasping claw 692 deployed via the first actuator 608 to reengage the holder 830.

The surgeon or assistant can cut the sutures 700 tying the prosthesis 810 to the holder 830 by applying a force to the second actuator 628 deploying the blade element 680 from the coupling end. The blade element 680 engages and cuts the sutures 700 as it travels distally into the cutting channel 834 of the holder 830 and rotationally around the claw sleeve 684, grasping claw 692, and the knob 872. The positioning of the second actuator 628 on the grip 606 can allow a surgeon or assistant to apply this force with a single finger and without necessitating adjustment of the handle 600 within his or her hand. The force can be removed from the second actuator 628, causing the blade element to retract into the holder engagement assembly 602. The holder 830 can then be withdrawn through the incision and from the surgical site with the handle 600.

Various features and advantages of the invention are set forth in the following claims.

What is claimed is:

1. A surgical apparatus for use with a prosthetic holder to which a prosthesis is secured by sutures, the apparatus comprising:
an elongated handle having a grip end and a coupler end, the handle defining a first aperture extending along a length from the grip end to the coupler end;
a first shaft at least partially disposed within the first aperture and having a first actuator at the grip end and a holder coupling mechanism at the coupler end; and
one of a second shaft and a sheath disposed along the handle and having a second actuator at the grip end and a cutting element at the coupler end;
wherein a force applied to the first actuator enables one of engagement and disengagement with the prosthesis holder, and a force applied to the second actuator enables deployment of a cutting element to cut the sutures securing the prosthesis to the prosthetic holder.

2. The surgical apparatus of claim 1, wherein the holder coupling mechanism comprises a grasping claw having a retracted state and an extended state.

3. The surgical apparatus of claim 2, wherein the holder coupling mechanism further comprises a claw sleeve for receiving the grasping claw in the retracted state.

4. The surgical apparatus of claim 1, further comprising a blade guard at least partially surrounding the cutting element.

5. The surgical apparatus of claim 4, wherein the blade guard includes a helical track and the cutter element includes a post received in the track such that when a force is applied to the second actuator, the cutter element rotates.

6. The surgical apparatus of claim 1, wherein the first shaft is resiliently biased toward the grip end of the handle.

7. The surgical apparatus of claim 1, wherein the one of a second shaft and a sheath is resiliently biased toward the grip end of the handle.

8. The surgical apparatus of claim 1, further comprising an actuator lock associated with the cutting element.

9. A surgical apparatus for use with a prosthesis, the apparatus comprising:
a prosthetic holder having a handle coupling element, the prosthetic holder coupled to a prosthesis by sutures;
an elongated handle having a grip end and a coupler end, the handle defining a first aperture extending along a length from the grip end to the coupler end;
a first shaft at least partially disposed within the first aperture and having a first actuator at the grip end and a holder coupling mechanism at the coupler end; and
one of a second shaft and a sheath disposed along the handle and having a second actuator at the grip end and a cutting element at the coupler end;
wherein a force applied to the first actuator enables one of engagement and disengagement of the holder coupling mechanism with the handle coupling element of the prosthesis holder, and a force applied to the second actuator enables deployment of a cutting element to cut the sutures securing the prosthesis to the prosthetic holder when the prosthesis holder is engaged.

10. The surgical apparatus of claim 9, wherein the prosthetic holder further comprises a branched retraction guide along which retaining faces move.

11. The surgical apparatus of claim 9, wherein the prosthetic holder further comprises a cutting channel positioned to receive the cutting element when the prosthesis holder is engaged.

12. The surgical apparatus of claim 9, wherein the first shaft is resiliently biased toward the grip end of the handle.

13. The surgical apparatus of claim 9, wherein the one of a second shaft and a sheath is resiliently biased toward the grip end of the handle.

14. The surgical apparatus of claim 9, further comprising an actuator lock associated with the cutting element.

15. The surgical apparatus of claim 9, wherein the handle includes a bend between the grip end and the coupler end.

16. The surgical apparatus of claim 9, further comprising a blade guard at least partially surrounding the cutting element.

17. The surgical apparatus of claim 16, wherein the blade guard includes a helical track and the cutter element includes a post received in the track such that when a force is applied to the second actuator, the cutter element rotates.

18. The surgical apparatus of claim 9, wherein the holder coupling mechanism comprises a grasping claw having a retracted state and an extended state.

19. The surgical apparatus of claim 18, wherein the holder coupling mechanism further comprises a claw sleeve for receiving the grasping claw in the retracted state.

20. A method of performing a surgical procedure, the method comprising:
providing a prosthetic holder and an elongated handle having a holder coupling mechanism and a cutting mechanism at a coupler end and a holder coupling actuator and a cutting actuator at a grip end;
tying a prosthesis to the prosthetic holder with sutures;
coupling the prosthetic holder to a handle by actuating the holder coupling mechanism;
positioning the prosthesis with the grip end of the handle;
decoupling the prosthetic holder from the handle by actuating the holder coupling mechanism;
suturing the prosthesis to the surrounding tissue;
recoupling the prosthetic holder from the handle by actuating the holder coupling mechanism; and
cutting the sutures by actuating the cutting mechanism.
